(19) United States
(12) Patent Application Publication
(10) Pub. No.: US 2019/0240492 A1
(43) Pub. Date: Aug. 8, 2019

(54) IMPLANTABLE MEDICAL DEVICE WITH A HYDROGEN GETTER

(71) Applicant: Cardiac Pacemakers, Inc., St. Paul, MN (US)

(72) Inventors: Michael C. Smith, Lino Lakes, MN (US); Gregory J. Sherwood, North Oaks, MN (US); Dustin J. Olynyk, Eagan, MN (US); John M. Edgell, Plymouth, MN (US); Daniel Bowen, Woodbury, MN (US); Scott Heineman, Vadnais Heights, MN (US); Joseph Thomas Delaney, JR., Minneapolis, MN (US); Kyle A. Kalstabakken, Apple Valley, MN (US)

(21) Appl. No.: 16/384,317

(22) Filed: Apr. 15, 2019

Related U.S. Application Data
(63) Continuation of application No. 14/700,831, filed on Apr. 30, 2015, now abandoned.
(60) Provisional application No. 61/989,720, filed on May 7, 2014.

(51) Int. Cl. A61N 1/375; H01M 10/42; A61N 1/378

(52) U.S. Cl. CPC A61N 1/375; H01M 2/1022; A61N 1/378; H01M 10/42

(57) ABSTRACT

This document describes an implantable medical device including a housing. Electronic components are located within the housing, and a non-metallic hydrogen getter is located within the housing.

### CLAIM OF PRIORITY

This application is a continuation of U.S. application Ser. No. 14/700,831, filed Apr. 30, 2015, which claims the benefit of priority under 35 U.S.C. § 119(e) of U.S. Provisional Patent Application Ser. No. 61/989,720, filed on May 7, 2014, each of which is herein incorporated by reference in its entirety.

### BACKGROUND

A pulse generator (implantable medical device or IMD), such as a cardiac rhythm management device, can include a sealed housing containing various electronic and electro-chemical components. Various hydrogen gas sources, such as heated insulation or circuit boards, electrochemical systems such as batteries or capacitors, and trapped hydrogen from previous processing or manufacturing can allow for the build up of hydrogen in the device.

Hydrogen gas has been shown to have potential deleterious effects on electrical components such as capacitors. For example, the capacitors can experience an increase in leakage current. U.S. Pat. No. 4,127,134 discusses a cardiac pacer with a palladium metal hydrogen getter included within the case of the cardiac pacer.

### OVERVIEW

In example 1, this document describes an implantable medical device including a housing. Electronic components are located within the housing, and a non-metallic hydrogen getter is located within the housing.

In example 2, the apparatus of example 1 can include a capacitor and a battery located within the housing.

In example 3, the apparatus of example 1, wherein the non-metallic hydrogen getter can include a hydrogen-absorbing polymer.

In example 4, the apparatus of example 3, wherein the hydrogen-absorbing polymer includes at least one of polyacetylene and polyvinyl acetylene.

In example 5, the apparatus of any of examples 1-4, wherein the non-metallic hydrogen getter includes an adhesive backing.

In example 6, the apparatus of any of examples 1-5, wherein the electronic components include a printed circuit board.

In example 7, the apparatus of example 6, further including one or more electronic components coupled to the printed circuit board and configured to perform signal analysis for providing electric therapy to a body, and further including a power supply coupled to the printed circuit board.

In example 8, the apparatus of any of example 1-7, wherein the hydrogen getter is sized to have a capacity of at least 100 μl.

In example 9, the apparatus of any of examples 1-8, wherein the hydrogen getter does not release H₂O as a byproduct.

In example 10, the apparatus of any of examples 1-9, wherein the housing includes a hermetically sealed housing.

In example 11, an apparatus includes an implantable medical device including a hermetically sealed housing; electronic components located within the housing, the electronic components configured to deliver electric therapy to a body; a battery located within the housing and connected to the electronic components; a capacitor located within the housing and connected to the electronic components; and a non-metallic hydrogen getter located within the housing.

In example 12, the apparatus of example 11, wherein the non-metallic hydrogen getter includes a hydrogen-absorbing polymer.

In example 13, the apparatus of example 12, wherein the hydrogen-absorbing polymer includes at least one of polyacetylene and polyvinyl acetylene.

In example 14, the apparatus of any of examples 11-13, further including one or more electronic components coupled to a printed circuit board within the housing and configured to perform signal analysis for providing the electric therapy to a body.

In example 15, the apparatus of any of examples 11-14, wherein the hydrogen getter is sized to have a capacity of at least 100 μl.

In example 16, the apparatus of any of examples 11-15, wherein the hydrogen getter does not release H₂O as a byproduct.

In example 17, a method includes providing an implantable medical device including a plurality of electronic components located within a housing; and placing a non-metallic hydrogen getter within the housing.

In example 18, the method of example 17, wherein the non-metallic hydrogen getter includes a hydrogen-absorbing polymer.

In example 19, the method of example 18, wherein the hydrogen-absorbing polymer includes at least one of polyacetylene and polyvinyl acetylene.

In example 20, the method of any of examples 17-19, wherein the hydrogen getter is sized to have a capacity of at least 100 μl.

### BRIEF DESCRIPTION OF THE FIGURES

The drawings illustrate generally, by way of an example, but not by a way of limitation, various embodiments discussed in the present document.

FIG. 1 shows an implantable medical device, according to an example.

FIG. 2 shows a hydrogen getter within the implantable medical device, according to an example.

FIG. 3 shows another view of the hydrogen getter within the implantable medical device, according to an example.

FIG. 4 shows another view of the hydrogen getter within the implantable medical device, according to an example.

FIG. 5 shows a top view of a hydrogen getter sheet before final preparation, according to an example.

FIG. 6 shows a top view of the hydrogen getter sheet of FIG. 5 formed into a plurality of individual hydrogen getters, according to an example.

FIG. 7 shows an individual hydrogen getter, according to an example.

### DETAILED DESCRIPTION

FIG. 1 shows an implantable medical device 100 in accordance with one example. The implantable medical device 100 includes a sealed metallic housing 110 and an attached header 120. The header 120 includes one or more ports 122 to receive a terminal pin 124 of an implantable lead 130. The lead 130 is configured to deliver pacing pulses, defibrillation shock energy, or cardioversion therapy to a heart, for example. The implantable medical device 100 can be implanted in a surgically-formed pocket in a patient's chest or other desired location. The implantable medical device 100 generally includes electronic components to perform signal analysis, processing, and control. The implantable medical device 100 can include a power supply such as a battery, a capacitor, and other components housed within housing 110. The implantable medical device 100 can include microprocessors mounted to circuit boards or flex circuits to provide processing and evaluation to determine and deliver electrical shocks and pulses of different energy levels and timing for ventricular defibrillation, cardioversion, and pacing to a heart in response to cardiac arrhythmia including fibrillation, tachycardia, and bradycardia via one or more electrodes of the lead 130.

The discussion herein can also apply to other types of implantable medical devices. For example, it can apply to implantable sensors that have a power system.

As noted above, there may be elevated levels of hydrogen gas in the implantable medical device. This hydrogen can be caused, for example, by hydrogen from a high voltage capacitor leaking from inside the capacitor into the device, hydrogen created by corrosion, pre-loaded hydrogen from the manufacturing process, hydrogen generated from other components, or multiple other factors that can vary over time. Again, hydrogen gas has been shown to have potential deleterious effects on electrical components of the device.

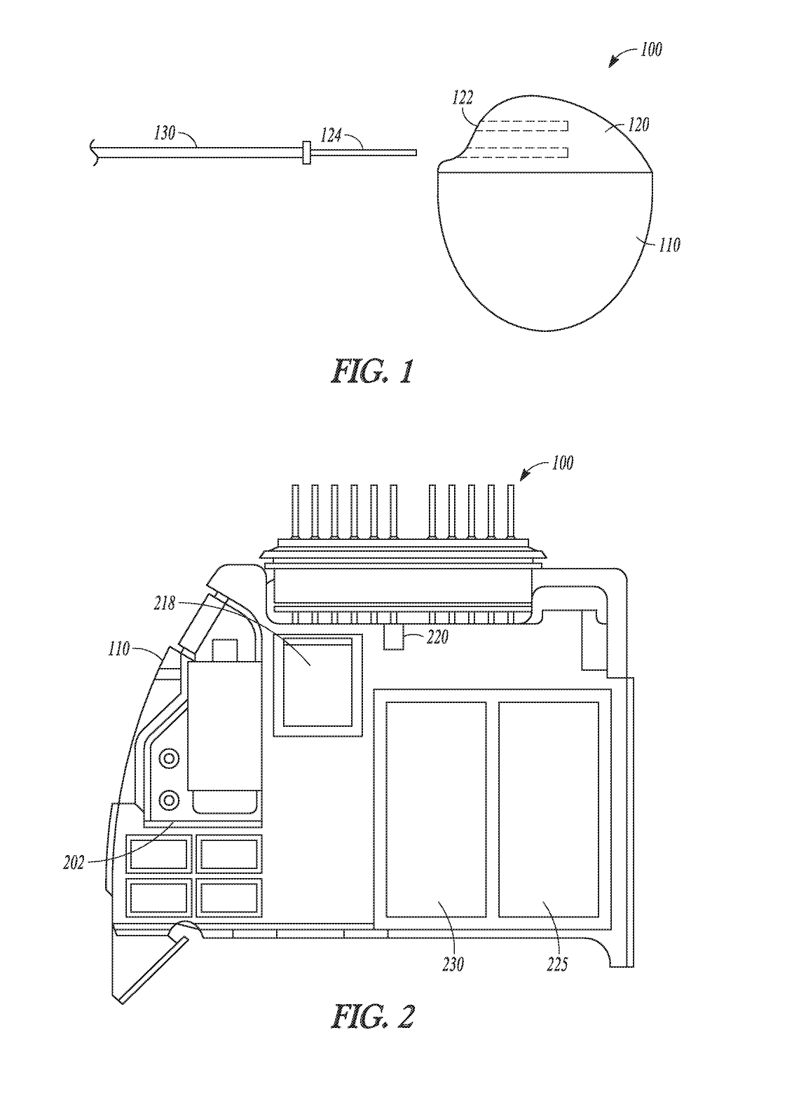

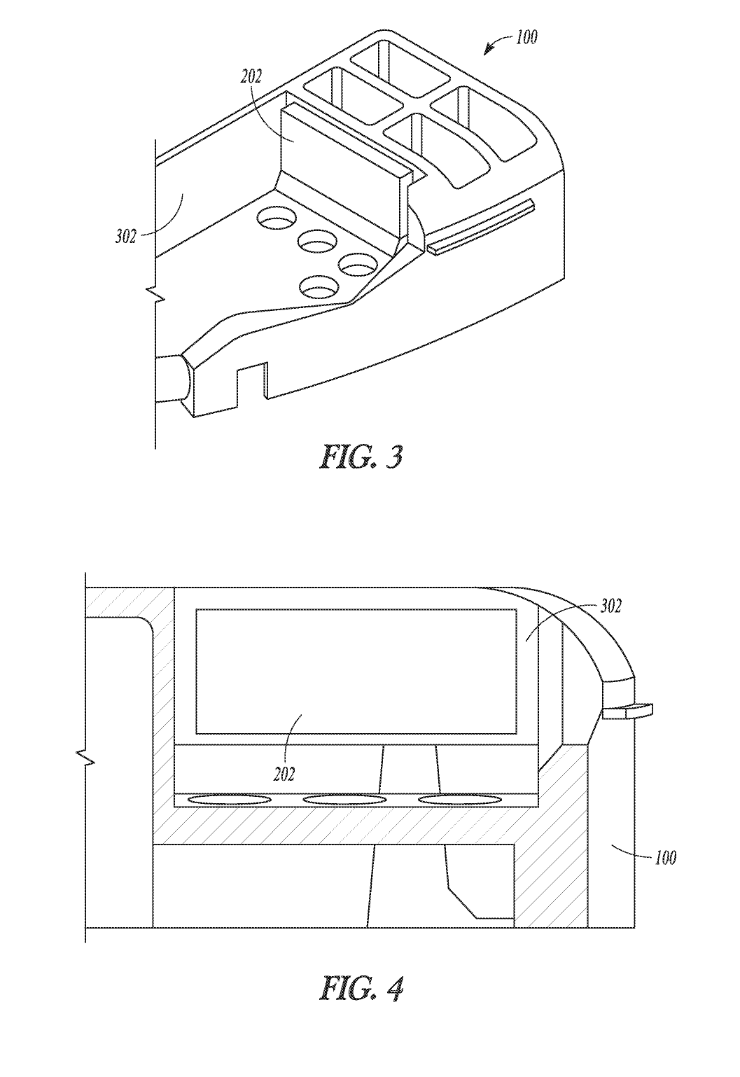

FIGS. 2-4 show examples of different components within the apparatus 100, including a hydrogen getter 202. The implantable medical device 100 can include the hermetically sealed housing 110. Within the housing 110 can be various electronic components 218 configured to perform signal analysis for providing the electric therapy to a body. For example, there can a printed circuit board 220, with microprocessors and other electronic components thereon. There can be electro-chemical devices within the housing such as a battery 225 located within the housing 110 and connected to the electronic components 218, and a capacitor 230 located within the housing 110 and connected to the electronic components 218. The hydrogen getter 202 is also mounted within the housing 210 and exposed to the inner environment of the housing.

In one example, hydrogen getter 202 is a non-metallic hydrogen getter, for example, made from a hydrogen absorbing polymer. The polymeric hydrogen getter 202 can be attached to the interior volume of the device and exposed to the gaseous space. The hydrogen gas reacts with the hydrogen getter 202 and the hydrogen getter 202 removes the hydrogen gas from the atmosphere of the device. The hydrogen getter 202 can be sized to provide ample capacity for anticipated hydrogen release. The hydrogen getter 202 can provide preventive protection to long term unknowns in the device and any other hydrogen generating sources. For example, in one embodiment, the hydrogen getter 202 is sized to have a capacity of at least 100 μl.

Some past hydrogen getters use the metal palladium as an active material. Potential problems from using palladium are that parts of the palladium metal may flake off the getter or the getter itself may become unattached from the housing. If that happens, deleterious effects to the electronics of the device may result.

Moreover, other past hydrogen getters work by absorbing hydrogen and releasing H₂O as a byproduct. Such a system then may require further desiccants to remove the H₂O. In contrast, the present polymeric hydrogen getter 202 does not produce H₂O as a byproduct. Accordingly, the polymeric, non-metallic, non-H₂O producing hydrogen getter 202 provides for a safer device.

In one example, the device 100 can include a liner 302, such as a plastic liner, that is located against some of the internal walls and other portions of the device. The hydrogen getter 202 can include an adhesive backing and be attached directly to the liner 302.

In some examples, it is desirable that the hydrogen getter 202 is small given the miniaturization of implantable electronics. Thus, using a hydrogen getter 202 with a hydrogen getter material with maximum absorption properties for a given mass of material is desirable.

One example is the hydrogen getter 202 can be formed of a processable, synthetically accessible polymer with as many unsaturated bonds as possible, thus acting as a smaller hydrogen sink. Some examples of such materials include polyisoprene, polybutadiene, polyvinyl propargyl ether, polyacetylene, and polyvinyl acetylene.

Polyacetylene and polyvinyl acetylene can be useful since they double the capacity of conventional hydrogen getters. For example, polyacetylene and polyvinyl acetylene have a wt-% H₂ sorption capacity of 3.73%.

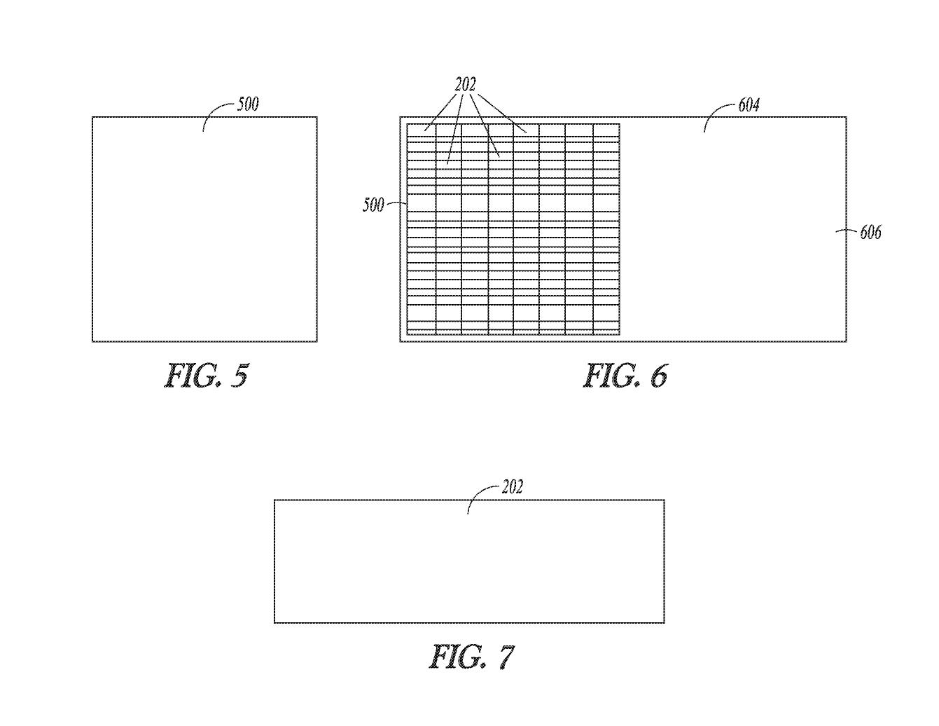

FIG. 5 shows a top view of a hydrogen getter sheet 500 before final preparation, according to an example; FIG. 6 shows a top view of the hydrogen getter sheet 500 formed into a plurality of individual hydrogen getters 202, according to an example; and FIG. 7 shows an individual hydrogen getter 202, according to an example.

Hydrogen getter sheet 500 can be formed from a hydrogen getter agent mixed with a polymeric material and formed into a relatively large sheet. For example, Vacuum Energy, Inc. provides a polymer hydrogen getter material. The large hydrogen getter sheet 500 then has an adhesive backing applied and is mounted to a liner 604. The hydrogen getter sheet 500 is then cut into a plurality of individual hydrogen getters 202. The liner 604 can include a blank area 606 to provide for ease of handing during manufacture and use.

Each individual getter 202 is then removed from the liner 604 and applied to the inside of an implantable device adhesively, as discussed above.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. The above description is intended to be illustrative, and not restrictive. The above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An apparatus comprising:
an implantable medical device including a housing, with a liner located against an internal wall of the housing;
electronic components located within the housing; and
a non-metallic hydrogen getter located within the housing, the non-metallic hydrogen getter configured to absorb hydrogen and form a bond with the hydrogen, wherein the non-metallic hydrogen getter includes an adhesive backing and is affixed to the liner.

2. The apparatus of claim 1, further including a capacitor and a battery located within the housing and connected to the electronic components.

3. The apparatus of claim 1, wherein the non-metallic hydrogen getter includes a hydrogen-absorbing polymer.

4. The apparatus of claim 3, wherein the hydrogen-absorbing polymer includes at least one of polyacetylene and polyvinyl acetylene.

5. The apparatus of claim 1, wherein the electronic components include a printed circuit board.

6. The apparatus of claim 5, wherein the electronic components include one or more electronic components coupled to the printed circuit board and configured to perform signal analysis for providing electric therapy to a body, and further including a power supply coupled to the printed circuit board.

7. The apparatus of claim 1, wherein the hydrogen getter is sized to have a capacity of at least 100 μl.

8. The apparatus of claim 1, wherein the hydrogen getter does not release H₂O as a byproduct.

9. The apparatus of claim 1, wherein the non-metallic hydrogen getter reacts with a hydrogen gas to remove the hydrogen gas from the atmosphere within the housing.

10. An apparatus comprising:
an implantable medical device including a hermetically sealed housing, with a liner located against an internal wall of the housing;
electronic components located within the housing, the electronic components configured to deliver electric therapy to a body;
a battery located within the housing and connected to the electronic components;
a capacitor located within the housing and connected to the electronic components; and
a non-metallic hydrogen getter located within the housing, the non-metallic hydrogen getter configured to absorb hydrogen and form a bond with the hydrogen, wherein the non-metallic hydrogen getter includes an adhesive backing and is affixed to the liner.

11. The apparatus of claim 10, wherein the non-metallic hydrogen getter includes a hydrogen-absorbing polymer.

12. The apparatus of claim 11, wherein the hydrogen-absorbing polymer includes at least one of polyacetylene and polyvinyl acetylene.

13. The apparatus of claim 10, further including a printed circuit board, and wherein the electronic components include one or more electronic components coupled to the printed circuit board within the housing and configured to perform signal analysis for providing the electric therapy to a body.

14. The apparatus of claim 10, wherein the hydrogen getter is sized to have a capacity of at least 100 μl.

15. The apparatus of claim 10, wherein the hydrogen getter does not release H₂O as a byproduct.

16. A method comprising:
providing an implantable medical device including a plurality of electronic components located within a housing, with a liner located against an internal wall of the housing; and
placing a non-metallic hydrogen getter within the housing, the non-metallic hydrogen getter configured to absorb hydrogen and form a bond with the hydrogen, wherein the non-metallic hydrogen getter includes an adhesive backing and is affixed to the liner.

17. The method of claim 16, wherein the non-metallic hydrogen getter includes a hydrogen-absorbing polymer.

18. The method of claim 17, wherein the hydrogen-absorbing polymer includes at least one of polyacetylene and polyvinyl acetylene.

19. The method of claim 16, wherein the hydrogen getter is sized to have a capacity of at least 100 μl.

20. The method of claim 16, wherein the hydrogen getter does not release H₂O as a byproduct.
